(19) United States
(12) Patent Application Publication
(10) Pub. No.: US 2025/0014698 A1
(43) Pub. Date: Jan. 9, 2025

(54) METHOD AND SYSTEM FOR ANALYSING MEDICAL IMAGES TO GENERATE A MEDICAL REPORT

(71) Applicant: EYETELLIGENCE LIMITED, Melbourne, Victoria 3000 (AU)

(72) Inventors: Zongyuan GE, Melbourne, Victoria (AU); Mingguang HE, Melbourne, Victoria (AU); Zhihong LIN, Melbourne, Victoria (AU); Wei MENG, Melbourne, Victoria (AU); Danli SHI, Melbourne, Victoria (AU)

(21) Appl. No.: 18/711,017

(22) PCT Filed: Nov. 17, 2022
(86) PCT No.: PCT/AU2022/051377

§ 371 Date: May 16, 2024

(30) Foreign Application Priority Data
Nov. 17, 2021 (AU) 2021903703

(51) Int. Cl. G16H 15/00; G06T 7/00; G06V 10/40

(52) U.S. Cl. CPC G16H 15/00; G06T 7/0012; G06V 10/40; G06T 2207/20084; G06T 2207/30041

(57) ABSTRACT

A system for analysing an image of a body part, the system including: an extractor module for extracting image features from the image; a transformer, including: an encoder including multiple encoder layers, and a decoder including multiple decoder layers, wherein each layer of the encoder and decoder includes a bi-linear multi-head attention layer configured to compute second-order interactions between vectors associated with the extracted image features; and a positional encoder configured to provide contextual order to an output of the bi-linear multi-head attention layer of the decoder; and a text-generation module to generate a text-based medical report of the image based on an output from the transformer.

### FIELD OF THE INVENTION

The present invention relates generally to analysing medical images and more specifically, to analysing images of body parts to generate a medical report. It will be convenient to describe in the invention in relation to the analysis of ophthalmic images, but it should be understood that the invention is not limited to that exemplary application.

### BACKGROUND

Convolutional Neural Network (CNN) based algorithm and product has been widely used for disease detection based on images. But it is only able to make a classification on a few pre-defined eye diseases (for example diabetic retinopathy, glaucoma and age-related macular degeneration) based on one single image modality e.g., full colour fundus photography.

Natural language text generation has been used in medical report generation for example for chest x-ray, using transformer-based captioning decoder and optimise the model with self-critical reinforcement learning.

However existing image analysis and medical report generating systems provide results that are inaccurate and are not broadly applicable to a wide variety of medical images.

It would therefore be desirable to provide a method and/or system for analysing an image of a body part that ameliorates and/or overcomes inconveniences of known methods and systems.

### SUMMARY OF THE INVENTION

According to a first aspect of the present invention, there is provided a system for analysing an image of a body part, the system including:
an extractor module for extracting image features from the image;
a transformer, including:
   an encoder including a plurality of encoder layers, and
   a decoder including a plurality of decoder layers,
   wherein each layer of the encoder and decoder comprise a bi-linear multi-head attention layer configured to compute second-order interactions between vectors associated with the extracted image features; and
   a positional encoder configured to provide contextual order to an output of the bi-linear multi-head attention layer of the decoder; and
a text-generation module to generate a text-based medical report of the image based on an output from the transformer.

In one or more embodiments, the bi-linear multi-head attention layer further comprises a bi-linear dot-product attention layer for producing one or more query vectors, key vectors and value vectors based on the extracted image features.

In one or more embodiments, the bi-linear multi-head attention layer is configured to compute the second-order interaction between the produced one or more query vectors, key vectors and value vectors.

In one or more embodiments, the positional encoder is based on periodic functions to describe relative location of medical terms in the medical report.

In one or more embodiments, the system further comprises an optimization module configured to perform recursive chain rule optimization of sentences in the text-based medical description.

In one or more embodiments, the positional encoder comprises a tensor having same shape as an input sequence.

In one or more embodiments, the encoder further comprises one or more add and learnable normalisation layers to produce combinations of possibilities of resulting features of the bi-linear multi-head attention layer.

In one or more embodiments, the encoder receives two or more inputs to contain feature representation from a plurality of image modalities.

In one or more embodiments, the system further comprises a search module configured to perform beam searching to further boost standardisation and quality of the generated medical reports.

In one or more embodiments, the text-generation module further comprises a linear layer and a Softmax function layer.

In one or more embodiments, the image of the body part is an ophthalmic image.

Another aspect of the invention provides a method for analysing an image of a body part, including the steps of:
using an extractor module to extracting image features from the image at an extractor module;
at transformer, including an encoder including a plurality of encoder layers, and a decoder including a plurality of decoder layers, using a bi-linear multi-head attention layer, forming part each layer of the encoder and decoder, to compute second-order interactions between vectors associated with the extracted image features;
using a positional encoder to provide contextual order to an output of the bi-linear multi-head attention layer of the decoder; and
using a text-generation module to generate a text-based medical report of the image based on an output from the transformer.

In one or more embodiments, the method further includes the step of:

using a bi-linear dot-product attention layer forming part of the bi-linear multi-head attention layer to producing one or more query vectors, key vectors and value vectors based on the extracted image features.

In one or more embodiments, the method further includes the step of:
using the bi-linear multi-head attention layer to compute the second-order interaction between the produced one or more query vectors, key vectors and value vectors.

In one or more embodiments, the method further includes the step of:
basing the positional encoder on periodic functions to describe relative location of medical terms in the medical report.

In one or more embodiments, the method further includes the step of:
using an optimization module to perform recursive chain rule optimization of sentences in the text-based medical description.

In one or more embodiments, the method further includes the step of:
using a tensor having same shape as an input sequence as part of the positional encoder.

In one or more embodiments, the method further includes the step of:
using one or more add and learnable normalisation layers to produce combinations of possibilities of resulting features of the bi-linear multi-head attention layer.

In one or more embodiments, the method further includes the step of:
using a search module configured to perform beam searching to further boost standardisation and quality of the generated medical reports.

In one or more embodiments, the method further includes the step of:
using a linear layer and a Softmax function layer as part of the text-generation module.

Aspects of the invention combine computer vision and natural language processing, and are able to generate text/sentence to name the eye diseases and pathologic lesions in various types of ophthalmic images.

Based on a database with images and text description for nearly 80 main type, 139 subtype of eye diseases (term) and >80 types of pathologic lesions (term), aspects of the invention provide a neural network architecture with attention mechanism to generate text that are in sentence structure logically interpretable in the norm of medical terminology.

Aspects of the invention provide a system that is able to generate text to clarify the image modality that is used to generate the image, generate text for the diagnosis of eye diseases and detection of pathologic lesions.

### BRIEF DESCRIPTION OF THE DRAWINGS

The invention will now be described in further detail by reference to the accompanying drawings. It is to be understood that the particularity of the drawings does not supersede the generality of the preceding description of the invention.

FIG. 1 is a schematic diagram of a system for analysing medical images according to an embodiment of the invention;

FIG. 2 is a schematic diagram of the operation of the system of FIG. 1, showing input images transformed into output textual medical report;

FIG. 3 is a flow chart showing steps carried out by an extractor forming part of the system shown in FIG. 1;

FIGS. 4 to 7 shows examples of feature maps with sizes of 56×56, 28×28, 14×14 and 7×7, visualising interesting regions where the network making decisions are based on, when medical images are input to the system of FIG. 1;

FIG. 8 is a schematic diagram showing various modules forming part of each encoder layer within an encoder, the encoder forming part of the system shown in FIG. 1;

FIG. 9 is a schematic diagram showing various modules forming part of each decoder layer within a decoder, the decoder forming part of the system shown in FIG. 1;

FIG. 10 is a schematic diagram showing layers within a bi-linear multi-head attention module forming part of each encoder later shown in FIG. 4 and each decoder later shown in FIG. 5;

FIG. 11 is a network architecture of bi-linear dot-product attention, which is a component used in a bi-linear multi-head attention module shown in FIG. 10;

FIG. 12 is a graphical representation of a positional encoding function applied to the decoder forming part of the system shown in FIG. 1;

FIG. 13 shows Stochastic Gradient Descent optimization process used in an embodiment of the invention to optimize the sequence of sentences in the generated medical report;

FIG. 14 illustrates an exemplary beam searching process implemented to further boost standardization and quality of generated medical reports;

FIG. 15 is a schematic diagram of one embodiment of an eye examination system including eye examination apparatus, the system of FIG. 1 forming part of the eye examination apparatus; and

FIG. 16 is a schematic diagram of a computer system forming part of the eye examination system of FIG. 9;

### DETAILED DESCRIPTION

### System

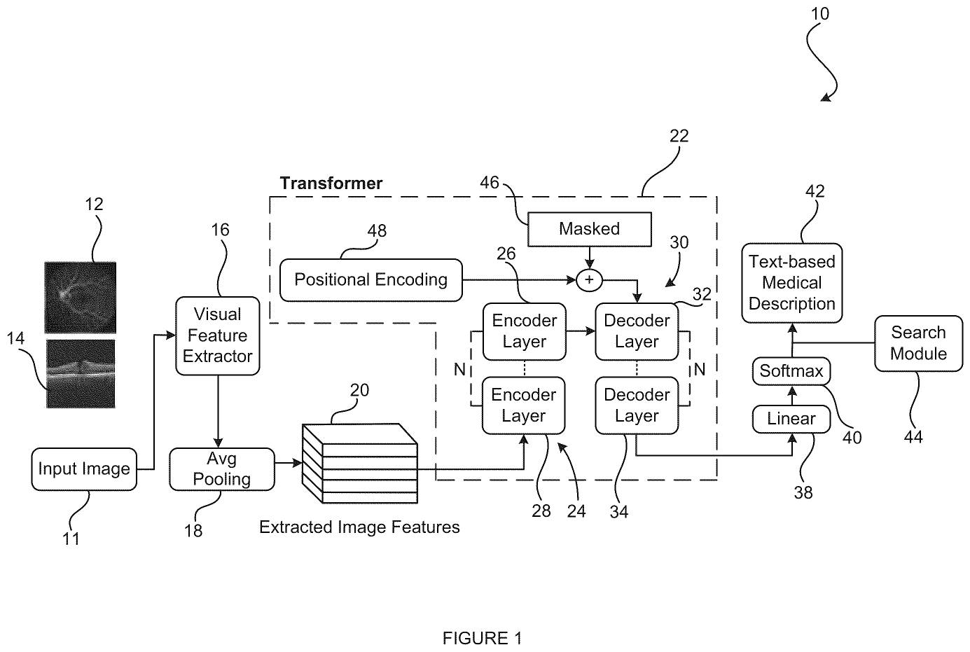

Referring now to FIG. 1, there is shown generally a system 10 for analysing medical images 11, such as exemplary ophthalmic images 12 and 14. The system 10 includes an extractor 16 to generate layers of extracted image features 20. An average pooling function 18 is applied to the extracted image features 20 which are then provided as an input to a transformer 22.

The transformer 22 includes an encoder 24 including multiple encoding layers, such as those layers referenced 26 and 28, that process the input received from the extracted image features 20 iteratively one layer after another. The transformer also includes a decoder 30, including multiple decoding layers, such as those layers referenced 32 and 34, that process an output received from the encoder 24 iteratively one layer after another.

The function of each encoder layer is to generate encodings that contain information about which parts of the inputs to the encoder 24 are relevant to each other. An attention mechanism is applied to describe a representation relationship between visual features.

Each encoder layer passes its encodings to the next encoder layer as inputs. Each decoder later does the opposite, taking all the encodings and using their incorporated contextual information to generate an output sequence-including a continuous sequential representation of the ophthalmic images—at the transformer output 36.

The output sequence from the transformer is provided to a linear layer 38 and then Softmax function layer 40 to generate a text-based medical report 42 comprising medical descriptions of each ophthalmic image.

Preferably, the system 10 further includes a search module 44 configured to perform beam searching to further boost standardisation and quality of the generated medical reports.

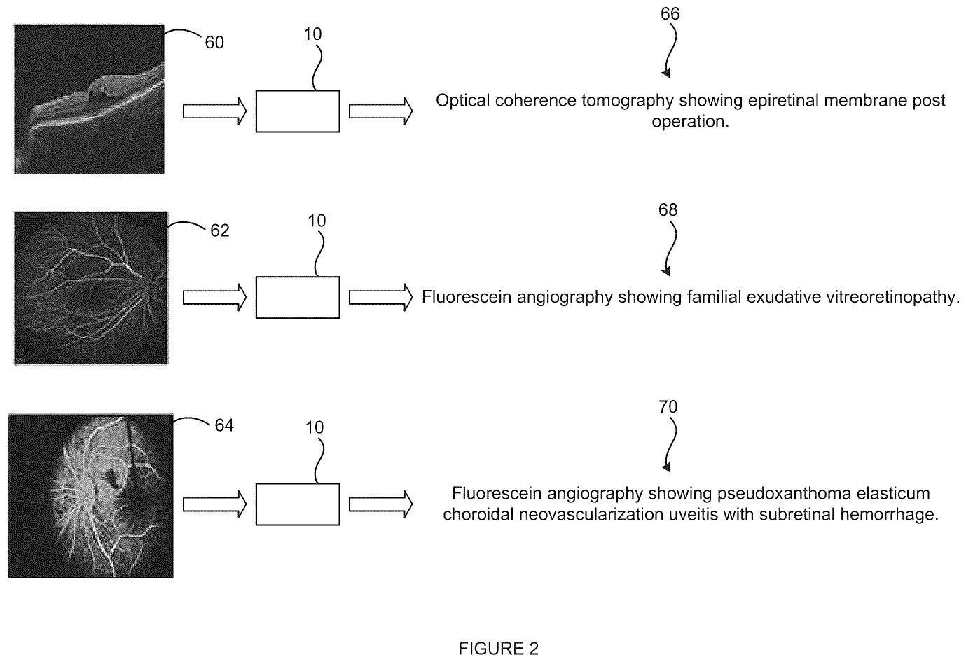

FIG. 2 shows three representative ophthalmic images 60, 62 and 64 as well as corresponding text-based medical descriptions 66, 68 and 70 generated by the system 10 for each image.

### Visual Extractor

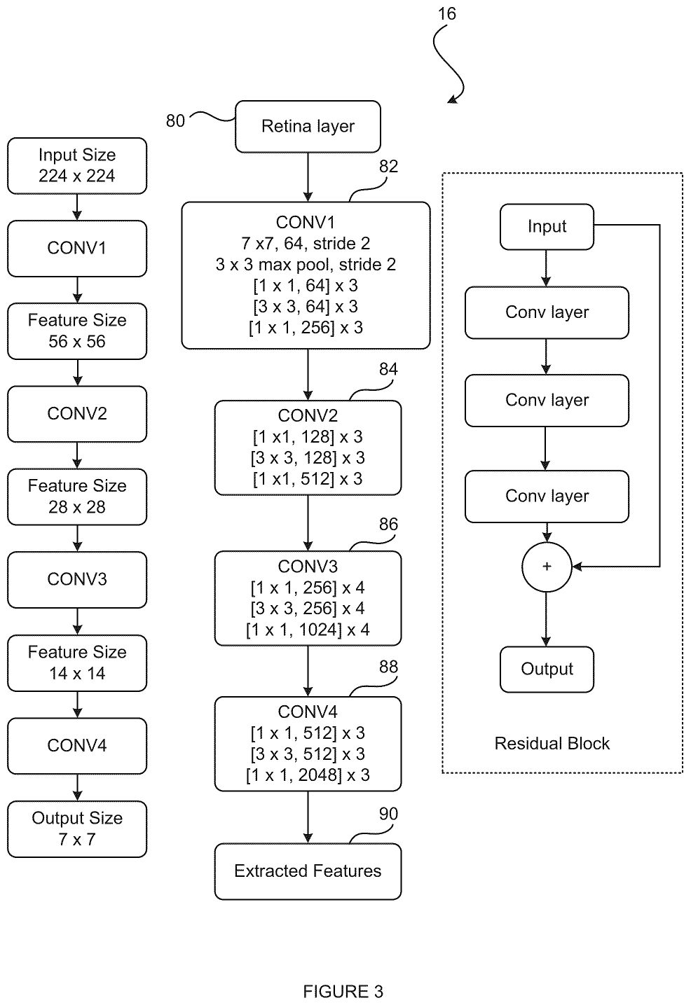

FIG. 3 depicts a sequence of operations performed by the extractor 16. Each medical image 80 (e.g., fundus images or OCT, etc.), is firstly pre-processed at step 80 to be resized to 256 by 256. The extractor 16 then extracts the visual image features prior to performing an average pooling operation and subsequently providing the extracted image features 20 to the transformer 22.

The extracted images features are vectors. The size of the vectors are determined by batch size, visual feature size (prior to the average pooling operation), and a predefined hidden feature dimension. The default number of predefined hidden feature dimension is 2048. Adjusting hidden feature dimension depends on the complexity and difficulty to generate unique visual features to represent different ophthalmic diseases. In other words, when there exists ophthalmic images with similar visual appearances but from different diseases, this feature dimension can be increased to a large number such as 4096.

The input ophthalmic images can be saved in various formats such as PNG, JPEG and TIFF. Information from images is processed into pixel-level vectors, by computer vision related libraries such Open-CV Python or Python Imaging Library. The sizes of pixel-level vectors are Width×Height×Color Channel. All images are resized to the same size to be used as inputs for the visual feature extractor 16.

In FIG. 3, there are shown four convolution block modules 82 to 88 in the visual feature extractor 16. Image feature maps are reduced after passing through each convolution module. The feature map output sizes of each of the four convolution modules 82 to 88 are 56×56, 28×28, 14×14 and 7×7 respectively. The repeated time is shown after the convolution kernel.

The conv1 module 82 includes three repeated residual blocks and each residual block 92 consists of three convolution operations 93, 94 and 95, with kernel sizes respectively of 1×1, 3×3 and 1×1, between input 96 and output 97. Similar to the conv1 module 82, conv2 module 84, conv3 module 86 and conv4 module 88 also have n (=3) repeated residual blocks and output feature channels are 512, 1024 and 2048. The 3×3 convolution operation is provided to ensure the visual receptive field and 1×1 convolution is provided to increase representative capability of the network in feature space. From conv1 module 82 to conv4 module 88, feature map sizes may be reduced and useful visual features can be extracted at step 90.

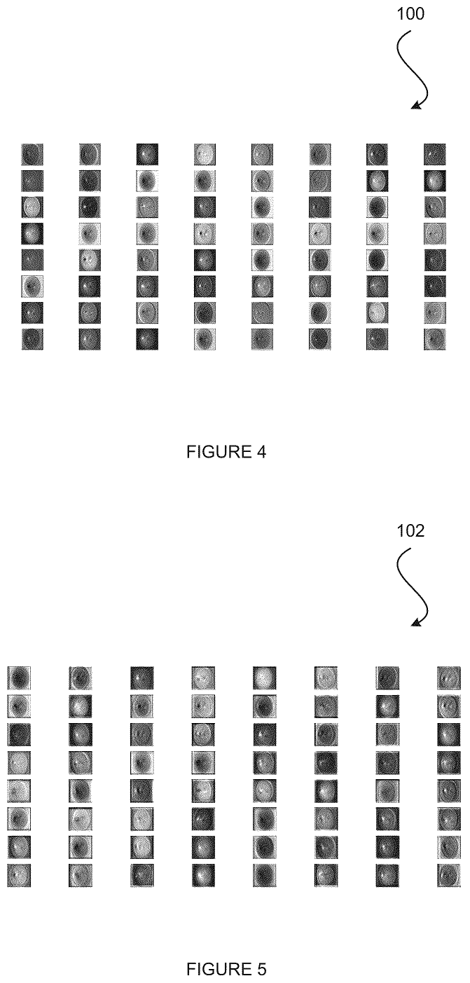

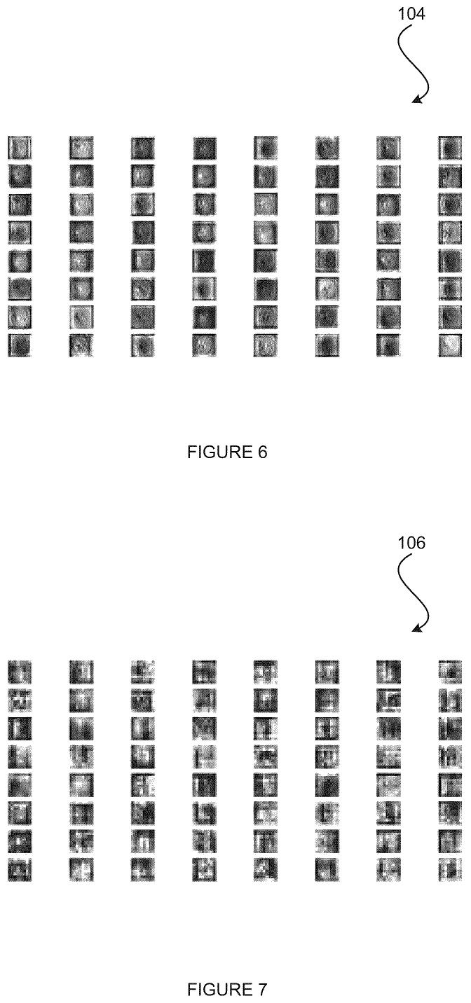

FIGS. 4 to 7 show examples of feature maps when ophthalmic images are input to the system of FIG. 1. Specifically, FIG. 4 shows an example of feature map 100 with size of 56×56, FIG. 5 shows an example of feature map 102 with size of 28×28, FIG. 6 shows an example of feature map 104 with size of 14×14 and FIG. 7 shows an example of feature map 106 with size of 7×7.

While the various aspects and embodiments are described with respect to ophthalmic images and using extractor 16 pretrained on a large-scale dataset such as Imagenet, it will be appreciated that analysis of medical images of other organs of the human body may also be performed by this invention. Currently, the extractor is by training a classification network of extractor with respective medical images as its inputs.

This extractor 16 is pretrained on a large-scale dataset to ensure representative capability of extracted features. In one embodiment, the extractor 16 may be formed by the ResNet101 classification network, even though other classification networks such as DenseNet and VGG are also suitable for use. One property of ResNet is a residual connection, which designs a shortcut for the input layer and sum operation of input identity and feature vectors processed by convolution layers. The difficulty of training deep neural networks is the vanishing gradient and design of residual connection minimises this difficulty by increasing information flow. The average pooling operation 18 is performed to reduce feature dimension.

### Encoder

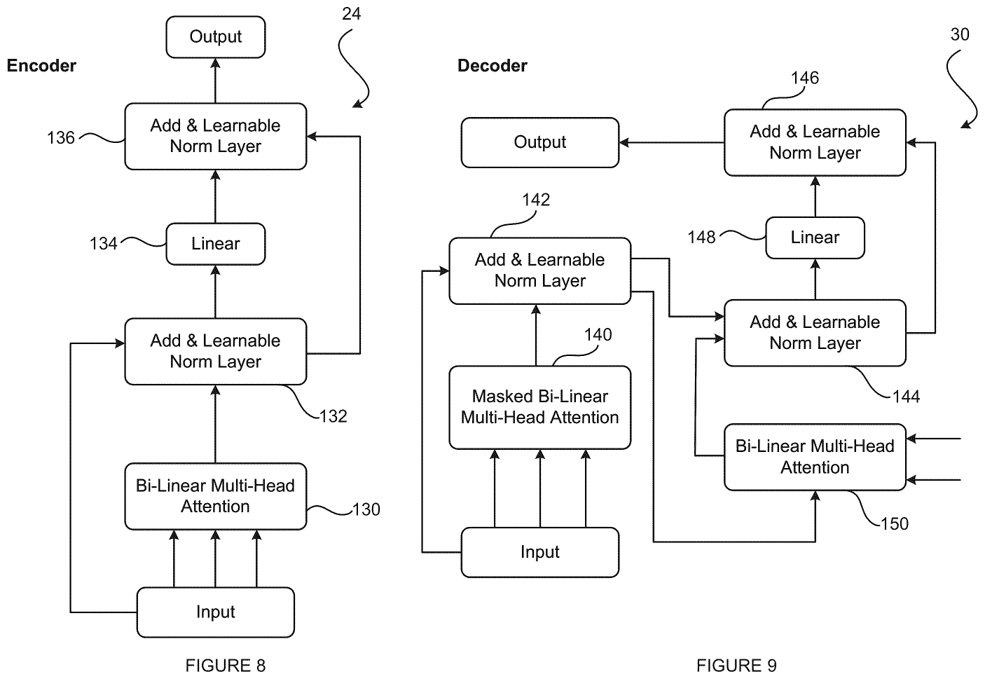

As can be seen in FIG. 8, the encoder 24 includes of a stack of N identical layers. Each layer of the encoder 24 includes an input 129, a Bi-Linear Multi-Head Attention Layer 130, a first Add and Learnable Normalisation (“Norm”) Layer 132, a Linear Layer 134, a second Add and Learnable Normalisation Layer 136 and an output 137.

The whole visual features are inputs of first encoder layer. The important part of visual features will be assigned a large attention weight. This invention is capable of working on various image modalities rather than the conventional single image modalities because of the design of the encoder. Unlike the conventional pretrained encoder, the encoder according to embodiments of this invention have multiple inputs to contain feature representation from several image modalities thereby making it robust to different modalities.

The add and normalisation layer reduces the information degradation by facilitating the information flow and the Learnable Normalisation Layer stabiles the training process. The function of the linear layer is to introduce more combination possibilities of learned features and a weighted relationship of previous features is learned. The Linear Layer can be understood as a convolution layer with the kernel size of 1.

Compared with the decoder 30 (described below), there is no bi-linear masked multi-head attention in the encoder 24.

The encoder 24 makes frequent usage of matrix multiplication in computations. The Bi-Linear Multi-Head Attention Layer 130 acts to improve the representative capability of intermediate features by providing second-order or higher-order interactions between the query, key-value matrices.

### Decoder

Each decoder layer 32, 34 consists of three major components: a self-attention mechanism, an attention mechanism over the encodings, and a feed-forward neural network. Each decoder layer functions in a similar fashion to the encoder, but an additional attention mechanism is inserted which instead draws relevant information from the encodings generated by the encoders.

Like the first encoder layer 28, the first decoder layer 32 takes positional information and embeddings of the output sequence as its input, rather than encodings. The transformer 22 can only use the current or previous generated words to predict next word which should appear in the sequence, so the output sequence is partially masked to prevent this reverse information flow. In other words, the whole sequence of sentences are inputs of transformer, sequences of sentence longer than current predicted sequences are masked to avoid transformer replying on ground truth of future words to make predictions.

The last decoder layer is followed by a final linear transformation layer 38 and Softmax layer 40, to produce the output probabilities over the vocabulary.

As can be seen in FIG. 9, the decoder 30 has the same number of layers as the encoder 24. Each decoder layer 32, 34 includes an input 149, a Masked Bi-Linear Multi-Head Attention Layer 140, Add and Learnable Norm Layers 142, 144 and 146, a Linear Layer 148, a Bi-Linear Multi-Head Attention Layer 150 and an output 151. The input of value and key vectors to the decoder 30 are the outputs from the encoder 34, and the query input is the output of the previous decoding layer. In other words, the input feature sizes of value or key can be different from the feature size of query without causing matrix multiplication incompatibilities in the self-attention.

Compared to the encoder, a Masked Bi-Linear Multi-Head Attention Layer 140 is introduced in the decoder 30. The function of the mask 46 in the decoder 30 is to prevent tokens in the future from being seen. The Masked Bi-Linear Muti-Head Attention Layer 140 is able to compute the relationship between visual features (key and value vectors) and language features (query vector). The Add and Learnable Norm Layers 142, 144 and 146 provide combination possibilities of resulting features of multi-head attention layer 140. The multi-head attention mechanism, which are applied in both the Masked Bi-Linear Muti-Head Attention Layer 140 and the Bi-Linear Muti-Head Attention Layer 150, employs a parallel version of attention function process.

The combination of an attention mechanism and positional encoding improves the efficiency of computations carried out by the decoder 24. With the positional encoding, the input sequential information can be processed as a whole rather than the sequential order. As a result, computations can be highly parallel in order to maintain an effective training time.

### Bi-Linear Dot-Product Attention Mechanism

The building blocks of the transformer 22 are scaled dot-product attention units. When extracted image features are passed into the transformer 22, attention weights are calculated between every token simultaneously. The attention units produce embeddings for every token in context that contain information about the token itself along with a weighted combination of other relevant tokens each weighted by its attention weight.

For each attention unit the transformer model learns three weight matrices: query weights, key weights and value weights. For each token, the input image feature embedding is multiplied with each of the three weight matrices to produce a query vector, a key vector and a value vector.

Attention weights are calculated using the query and key vectors: each attention weight is the dot product between a query vector and a key vector. The attention weights are divided by the square root of the dimension of the key vectors, which stabilizes gradients during training, and passed through a Softmax layer which normalizes the weights. The output of the attention unit for token i is the weighted sum of the value vectors of all tokens, weighted by the attention to each token.

The attention calculation for all tokens can be expressed as one large matrix calculation using the Softmax function, which is useful for training due to computational matrix operation optimizations that quickly compute matrix operations.

The attention mechanism in the decoder 30 is more complex in comparison to the attention mechanism in the encoder 24. The query, key, value vectors in the bilinear masked multi-head attention module are the same, while the query, key, value vectors in the bilinear masked multi-head attention module are different.

The inputs of the bilinear masked multi-head attention module 130 appearing in the encoder 24 are different from inputs of the bilinear multi-head attention module 150 in the decoder 30. In other words, the query, key and value vectors as inputs of this attention module in the encoder 24 are all the same, while the inputs in decoder 30 are different by processing language-related features with the query vector and visual features with key and value vectors.

There are feature dimension differences between medical images and diagnostic reports. It is challenging and difficult to associate regions of interest in the medical images with feature maps of corresponding reports. The overall architecture of Bi-Linear Dot-Attention mechanism involves interaction between query, key and value.

The bi-linear dot-product attention, which describes the mapping relationship between the query matrix and key-value matrices, is defined as follows:

$$Bi - Linear Attention ( K , Q , V ) = ⊗ ( ⊗ ( Q {V}_{r e f i n e} , {KQ}_{r e f i n e} ) , K )$$

$${K}^{′} = Relu ( N {N}_{1} ( K ) ) , {V}^{′} = Relu ( N {N}_{5} ( V ) )$$

$${Q}_{1}^{′} = Relu ( N {N}_{2} ( R e l u ( N {N}_{3} ( Q ) ) ) ) , {Q}_{2}^{′} = Relu ( N {N}_{4} ( Q ) )$$

$$Q {V}_{r e f i n e} = ⊗ ( {Q}_{2}^{′} , {V}^{′ T} )$$

$$K {Q}_{r e f i n e} = softmax ( ⊗ ( {Q}_{1}^{′} , {V}^{′ T} ) )$$

where K, Q, V, NN and ⊗ represent key matrix, query matrix, value matrix, linear layer and element-wise matrix multiplication.

### Bi-Linear Multi-Head Attention Mechanism

One set of matrices of query weights, key weights and value weights is called an attention head, and each layer in a transformer 22 has multiple attention heads. While each attention head attends to the tokens that are relevant to each token, with multiple attention heads the model can do this for different definitions of “relevance”. In addition, the influence field representing relevance can become progressively dilated in successive layers. The influence field of a single layer can be understood as the matrices relationship learned by attention mechanism inside a single head. The whole transformer architecture usually contains several layers than a single layer. The weighted relationship of query, key and value of previous layers influences later layers. The above relationship is denoted as influence field which describes a representation of output using the input with sequential information.

Many transformer attention heads encode relevance relations that are meaningful to humans. The computations for each attention head can be performed in parallel, which allows for fast processing. The outputs for the attention layer are concatenated to pass into the feed-forward neural network layers.

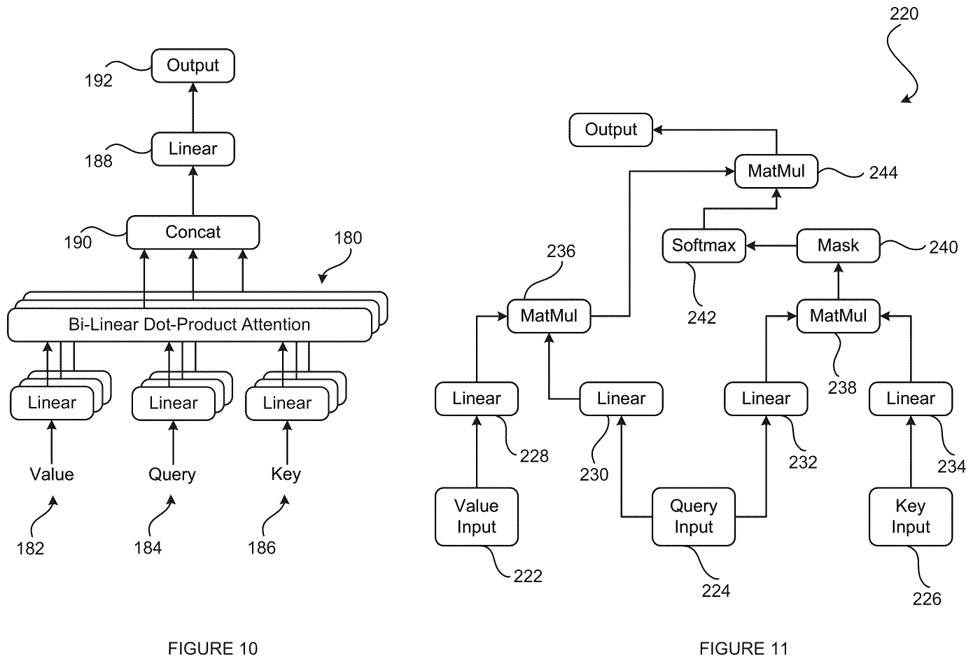

The design of the Bi-Linear Multi-Head Attention Layers is depicted in FIG. 10, and is intended to improve the feature representation capability in the subspace. The computation of Bi-Linear Multi-Head Attention is defined as:

$$Bi - Linear MultiHead ( K , Q , V ) = Concat ( {head}_{1} , … , {head}_{1} )$$

$$head = Bi - Linear MultiHead ( K , Q , V )$$

Bi-linear multi-head attention is a combination of single bi-linear head attention. The parameter, the number of heads, can be adjusted to achieve different representation subspaces. The choice of this parameter should depend on complexity of representing retina images, its corresponding medical reports and relationships between retina images and reports in feature spaces. To balance the computation time required and representative feature space, the hidden size of each bi-linear attention head can be reduced.

Referring to FIG. 10, the inputs of the bi-linear multi-head attention 180 are value, query and key vectors 182 to 186. A linear layer 188 controls the channel number of hidden features. The bi-linear dot-product attention 180 is an attention mechanism involving second-order interaction. The number of heads further increase the representative capability of each bi-linear dot-product attention module. The function of multi-head is to provide feature space variance. A concatenate operation 190 forms a new feature set later projected by the Linear layer 188, prior to the output 192.

The Bi-Linear Multi-Head Attention Layer 150 is to conduct self-attention to produce a diverse representative space. The inputs of the bi-linear multi-head attention layer 150 are same as conventional multi-head attention layers, and differences between them are computations of attention mechanism. Conventional attention mechanisms only compute the first-order interaction with matrix multiplication between query, key and value matrices, but the Bi-Linear Multi-head Attention Layer 150 computes the second-order interaction.

The inputs of first bi-linear multi-headed attention layer 150 are the outputs of extracted visual features, so that the visual extractor and encoder are connected in series. The above bi-linear multi-head attention can also be applied to non-ophthalmic images, but non-ophthalmic images might not require such strong attention interaction to describe the visual feature representation. To distinguish the visual image differences like the dog and cat, the conventional first attention mechanism should be enough.

FIG. 11 is a network architecture 220 of the bi-linear dot-product attention 180, which is a component used in a bi-linear multi-head attention module shown in FIG. 10. Value input 222, query input 224 and key input 226 represent three different input matrices of the bilinear attention module. A linear layer, including linear transformation units 228 to 234, applies a linear transformation to input data from the value input 222, query input 224 and key input 226 and controls the output channel number of features.

Outputs from the linear transformation units 228 to 234 are applied to MatMul units 236 and 238. The MatMul units 236 and 238 each have 2 inputs (A with the dimension of m×n and B with the dimension of o×p). If the dimension sizes of input A and input B are identical, each MatMul unit denotes element-wise matrix multiplication. If second dimension n of first input A matches first dimension o of second input B, each Matmul unit denotes dot-product matrix multiplication. There are 3 matrix multiplication operations to introduce high-order interactions.

A mask function 240 and Softmax function 242 are applied to the output of the Matmul unit 238. The Softmax function normalises K probabilities distribution proportional to the exponential of input probability distributions. After applying the Softmax operation, the summation of all normalised exponential of input probability distributions is equal to 1.

The mask operation is to prevent the neural network from cheating to make predictions based on the ground truth (words appearing in the future) rather than visual cues and current predicted result. The mask operation is to fill the upper triangle of targeted matrix with extremely low values and keep values below diagonal line constant.

Finally, the output of the Softmax function 242 and the output of the MatMul function 236 are applied as inputs to a MatMul function 244 prior to an output 245.

### Positional Encoding

In transformer architecture, positional encoding is used to give the order context to the non-recurrent architecture of multi-head attention. When the recurrent networks are fed with sequence inputs, the sequential order (ordering of time-steps) is implicitly defined by the input. However, the Multi-Head Attention layers in a transformer are a feed-forward layers and reads a whole sequence at once. As the attention is computed on each datapoint (time-step) independently, the context of ordering between data points is lost and the attention is invariant to the sequence order. The same is generally true for other non-recurrent architectures like convolutional layers where only a small sequential ordering context is present, limited by the size of the convolution kernel.

To alleviate this problem, the concept of positional encoding is used. This involves adding a tensor (of the same shape as the input sequence) with specific properties to the input sequence. The positional encoding tensor is chosen such that the value difference of the specific steps in the sequence correlates to the distance of individual steps in time (sequence). Positional encoding is based on periodic functions, which have the same value at regular intervals. Sine and cosine functions are implemented as periodic functions of positional encoding to describe relative location of medical terms in the medical reports.

Conventional transformers requires positional encoding for both encoder and decoder, and are suitable for the sequence-to-sequence task such as machine translation. Compared with conventional transformers, the system 10 targets the image-to-sentence translation, and so the positional encoding is redundant for the encoder 24 of the transformer 22. Accordingly, positional encoding is only applied to the decoder 30 of the transformer 22.

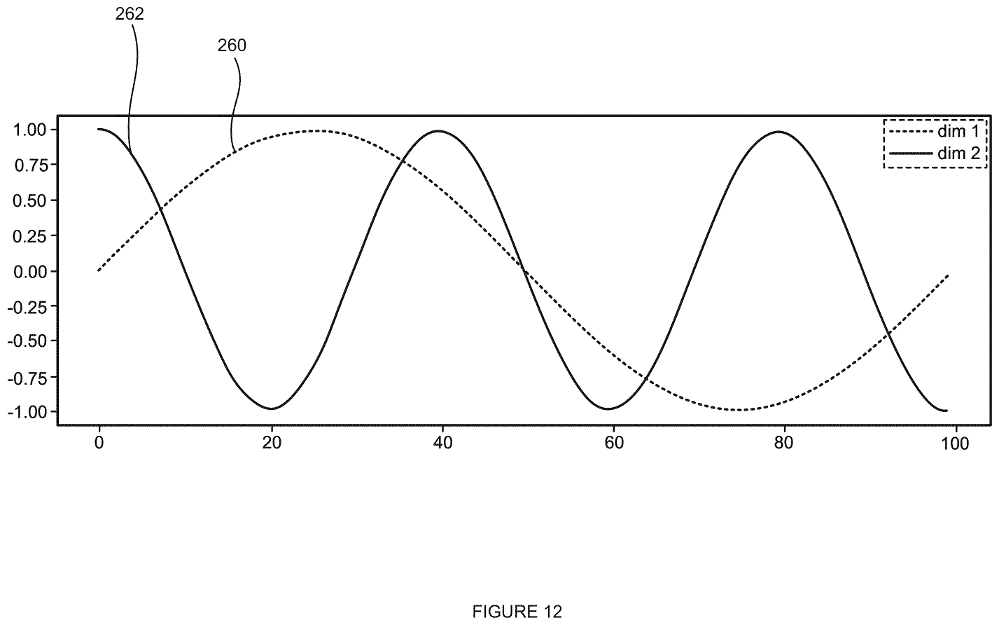

A graphical representation of the positional encoding function is shown in FIG. 12. In order to reflect the sequential information, output of a positional encoder 48 is the summation of input sequential features and results of periodic functions.

The positional encoding function of the positional encoder 48 is defined as:

$$positional encoding ( loc , 2 x ) = {sin ( loc / 10000 )}^{2 x / sf}$$

$$positional encoding ( loc , 2 x + 1 ) = cos {( loc / 10000 )}^{2 x / sf}$$

where x is the location in sequence implementation; i is the dimension and sf is the dimension size of input sequential features. In other words, the encoded positional vectors are different along different dimensions.

FIG. 12 shows two periodic curves 260 and 262 that represent the visualisation of positional encoding along different dimensions to represent location in the sequence. The dimension size of the first periodic curve 260 is 1 and the dimension size of second periodic curve 262 is 2.

The optimization process of the system 10 is formulated as a recursive chain rule of generating sequences. Common optimization algorithms include Stochastic Gradient Descent, Adadelta, RMSprop and Adam. The Adam optimizer is selected for use in the system 10 rather than Stochastic Gradient Descent because Stochastic Gradient Descent is more likely to be trapped in the local minimum. The computation of adaptive moment estimation required to initialize with first moment vector, second moment vector and timestep. Adam can be understood as an advanced version of Stochastic Gradient Descent, which also computes stostatic gradients at the beginning. The biased first and second moment estimation is updated, and then corresponding biased-corrected moment estimation are computed. During the optimization process, the gradient clipping is implemented to avoid the gradient explosion.

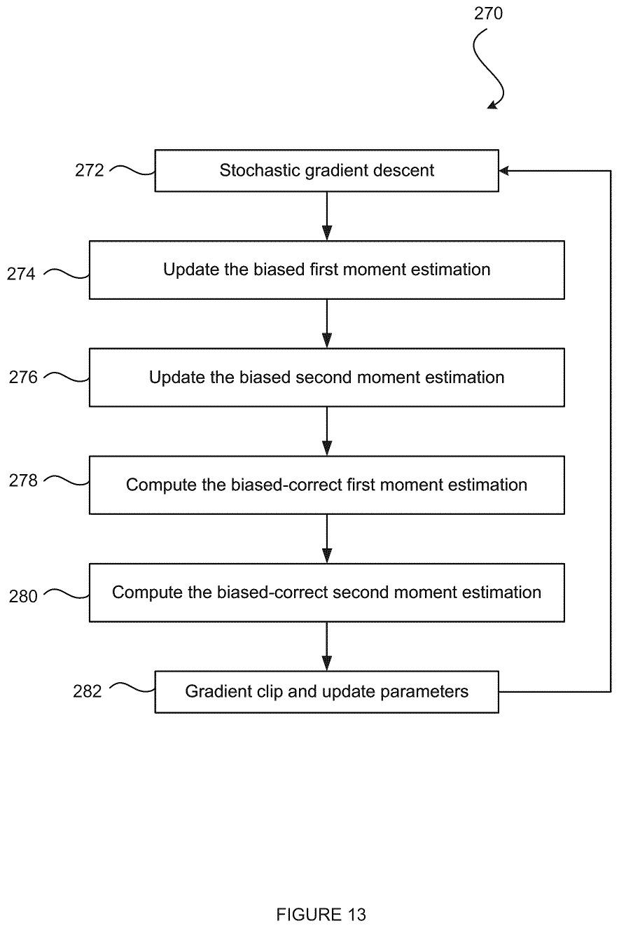

FIG. 13 illustrates an exemplary beam searching process 270 implemented by the system 10 to further boost standardization and quality of generated medical reports. The goal of the medical report generation task is to produce a sequence which is able to describe the clinical impression shown an input image. Predicted outputs are sequences rather than simple classification results. At steps 272 to 282, a sequence of probabilities is maximized, and sequences of probabilities are computed by multiplying the candidate probability together.

Moreover, the system 10 implements a beam searching algorithm which defines the beam size, which is the number of beams for parallel searching. Greedy search algorithm is a special case of beam searching algorithm which only selects the best candidate at each time step, and this might result in a local optimal choice rather than the global optimal choice. Supposed that the beam size is k, the beam searching can be categorised into following steps. To begin with, the top k words with the highest probabilities are chosen as k parallel beams. Next, k best pairs including first and second word are computed by comparing conditional probability. Finally, this process is repeated until a stopping token appears.

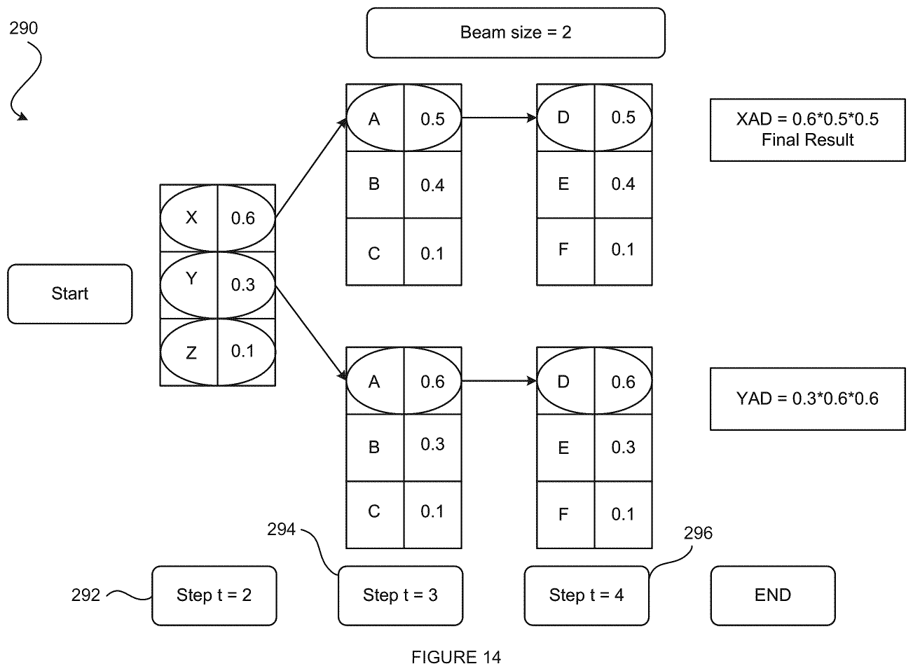

FIG. 14 illustrates such a beam searching process 290 implemented by the system 10. In this exemplary diagram, the beam size is set to 2. The capital letters of the alphabet represent the candidate pool from which selection is made. The corresponding appearance probability of each capital letter is shown next to the capital letter. The arrow indicates the sequence generation direction. The circle shows the candidate selected at each of steps 292, 294 and 296.

Examples of ophthalmic diseases that can be assessed via the medical reports include, but are not limited to, astrocytoma, macular hole, choroidal folds, retinal dystrophy, choroidal hemangioma, eales peripheral vasculitis, retinal edema, choroidal melanoma, age-related macular degeneration, melanocytoma, purtscher's retinopathy, rpe detachment, congenital hypertrophy of the retinal pigment epithelium, rpe tear, post pan retinal photocoagulation, hypertensive retinopathy, optic disc edema, von hippel lindau, hamartoma, myopia, retinal telangiectasia, choroideremia, retinal vein occlusion, infection, proliferative vitreoretinopathy, choroiditis, neuroretinitis, choroidal nevus, glaucoma, diffuse unilateral subacute neuroretinitis, post operation, vitritis, vogt-koyanagi-harada, and neuroretinitis, optic disc drusen, vasculitis, myelinated nerve fiber, idiopathic retinitis, coloboma, optic neuropathy, crystalline retinopathy, retinal neovascularization, systemic lupus erythematosus, coats retinal telangiectasia, cystoid macular edema, choroidal metastasis, retinal detachment, persistence and hyperplasia of the primary vitreous, central serous chorioretinopathy, vitreomacular traction, post retinal photocoagulation, epiretinal membrane, angioid streak, vasculitis, tuberous sclerosis, aneurysms, retinal macroaneurysm, diabetic retinopathy, macular edema, macular dystrophy, artery occlusion, pseudoxanthoma elasticum, uveitis, bull's eye maculopathy, gyrate atrophy, retinopathy of prematurity, optic nerve pit, dry age-related macular degeneration, familial exudative vitreoretinopathy, chloroquine toxicity, birdshot chorioretinopathy, posterior vitreous detachment, choroidal osteoma, choroidal neovascularization, morning glory syndrome, sarcoidosis, asteroid hyalosis, terson's syndrome, white dot syndrome.

### Implementation in Eye Examination Machine

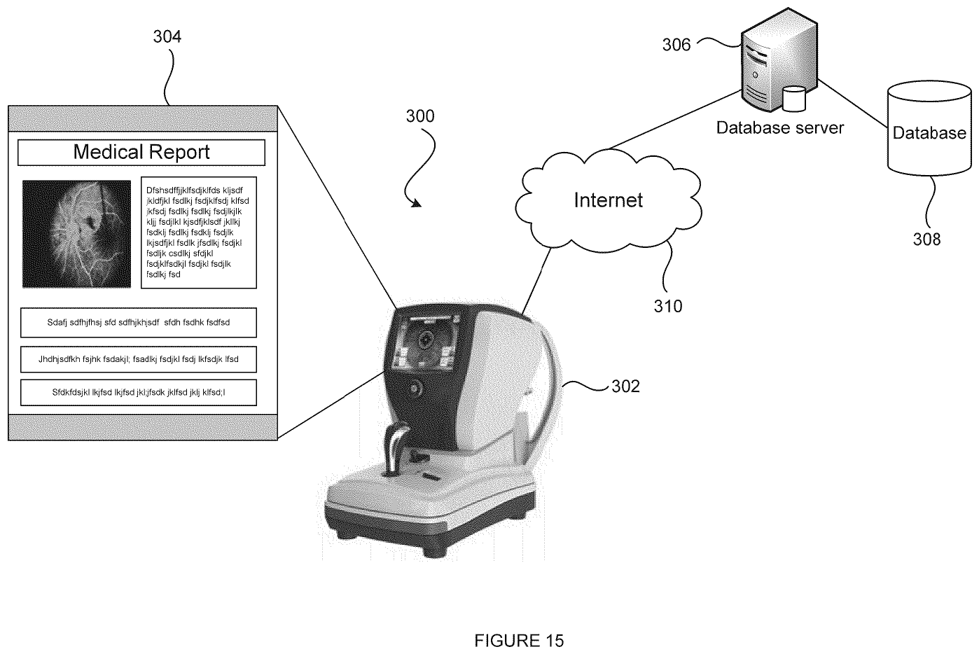

Referring to FIG. 15, there is shown an embodiment in which the system 10 for analysing ophthalmic images forms part of an eye examination system 300. The eye examination system 300 includes an eye examination equipment 302, such as an opthalmoscope, retinascope or retinal camera, providing a graphic user display 304, and a database 308 in communication with a database server 306. The eye examination equipment 302 and server 306 are interconnected by means of the Internet 310 or any other suitable communications network.

Ophthalmic images captured by the eye examination equipment 302 and data that may be accessed by the eye examination equipment 302 to enable the system 10 to perform the above-described functionality are maintained remotely in the database 308 and may be accessed by an operator of the eye examination equipment 302. Whilst in this embodiment of the invention the items are maintained remotely in database 308, it will be appreciated that the items may also be made accessible to the eye examination equipment 302 in any other convenient form, such as a local data storage device.

The eye examination equipment 302 may be implemented using hardware, software or a combination thereof and may be implemented in one or more computer systems or processing systems. In particular, the functionality of the eye examination equipment 302 and its graphic user display 304, as well as the server 306 may be provided by one or more computer systems capable of carrying out the above-described functionality.

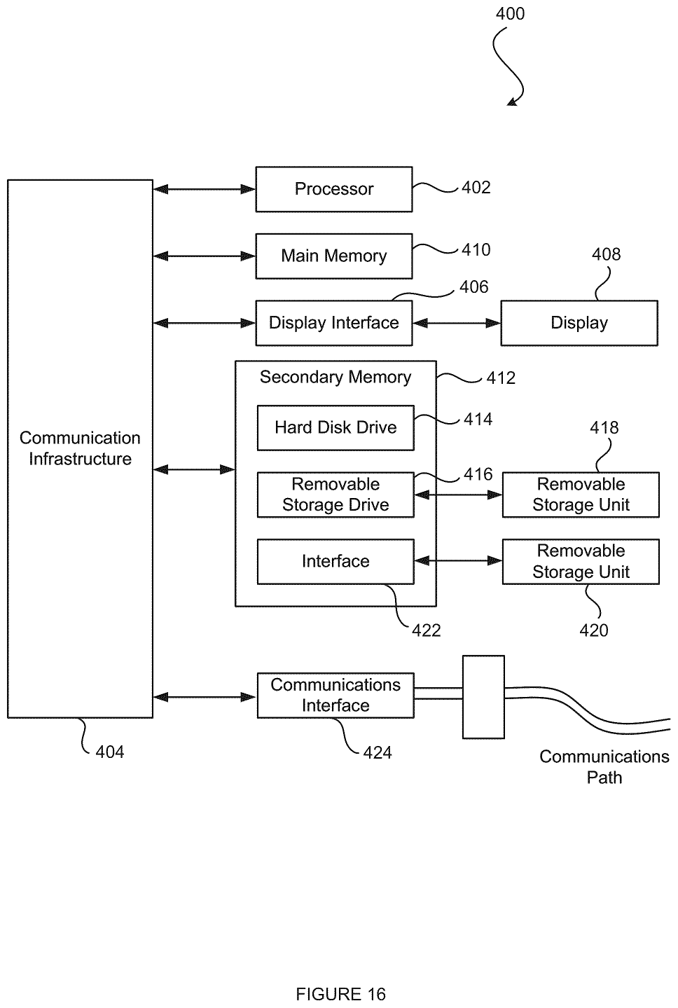

An exemplary computer system 400 is shown in FIG. 16. The computer system 400 includes one or more processors, such as processor 402. The processor 402 is connected to a communication infrastructure 404. The computer system 400 may include a display interface 406 that forwards graphics, texts and other data from the communication infrastructure 404 for supply to the display unit 408. The computer system 400 may also include a main memory 410, preferably random access memory, and may also include a secondary memory 412.

The secondary memory 412 may include, for example, a hard disk drive 414, magnetic tape drive, optical disk drive, etc. The removable storage drive 416 reads from and/or writes to a removable storage unit 418 in a well known manner. The removable storage unit 418 represents a floppy disk, magnetic tape, optical disk, etc.

As will be appreciated, the removable storage unit 418 includes a computer usable storage medium having stored therein computer software in a form of a series of instructions to cause the processor 402 to carry out desired functionality. In alternative embodiments, the secondary memory 412 may include other similar means for allowing computer programs or instructions to be loaded into the computer system 400. Such means may include, for example, a removable storage unit 420 and interface 422.

The computer system 400 may also include a communications interface 424. Communications interface 424 allow software and data to be transferred between the computer system 400 and external devices. Examples of communication interface 424 may include a modem, a network interface, a communications port, a PCMIA slot and card etc. Software and data transferred via a communications interface 424 are in the form of signals which may be electromagnetic, electronic, optical or other signals capable of being received by the communications interface 424. The signals are provided to communications interface 424 via a communications path such as a wire or cable, fibre optics, phone line, cellular phone link, radio frequency or other communications channels.

Although in the above-described embodiments the invention is implemented primarily using computer software, in other embodiments the invention may be implemented primarily in hardware using, for example, hardware components such as an application specific integrated circuit (ASICs). Implementation of a hardware state machine so as to perform the functions described herein will be apparent to persons skilled in the relevant art. In other embodiments, the invention may be implemented using a combination of both hardware and software.

While the invention has been described in conjunction with a limited number of embodiments, it will be appreciated by those skilled in the art that many alternative, modifications and variations in light of the foregoing description are possible. Accordingly, the present invention is intended to embrace all such alternative, modifications and variations as may fall within the spirit and scope of the invention as disclosed.

1. A system for analysing an image of a body part, the system comprising:
an extractor module for extracting image features from the image;
a transformer including:
an encoder including a plurality of encoder layers, and
a decoder including a plurality of decoder layers,
wherein each layer of the encoder and decoder includes a bi-linear multi-head attention layer configured to compute second-order interactions between vectors associated with the extracted image features; and
a positional encoder configured to provide contextual order to decoder bi-linear multi-head attention layer output; and
a text-generation module configured to generate a text-based medical report of the image based on an output from the transformer.

2. The system of claim 1, wherein each bi-linear multi-head attention layer further includes a bi-linear dot-product attention layer for producing one or more query vectors, key vectors and value vectors based on the extracted image features.

3. The system of claim 2, wherein the each bi-linear multi-head attention layer is configured to compute the second-order interaction between the produced one or more query vectors, key vectors and value vectors.

4. The system of claim 1, wherein the positional encoder is based on periodic functions to describe relative location of medical terms in the medical report.

5. The system of claim 1, further comprising an optimization module configured to perform recursive chain rule optimization of sentences in the text-based medical description.

6. The system of claim 1, wherein the positional encoder comprises a tensor having a same shape as an input sequence.

7. The system of claim 1, wherein the encoder further comprises one or more add and learnable normalisation layers to produce combinations of possibilities of resulting features of each of the bi-linear multi-head attention layer included in the encoder.

8. The system of claim 1, wherein the encoder receives two or more inputs to contain feature representation from a plurality of image modalities.

9. The system of claim 1, further comprising a search module configured to perform beam searching to further boost standardisation and quality of the generated medical reports.

10. The system of claim 1, wherein the text-generation module further comprises a linear layer and a Softmax function layer.

11. The system according to claim 1, wherein the image of the body part is an ophthalmic image.

12. A method for analysing an image of a body part, the method comprising:
using an extractor module to extracting image features from the image at an extractor module;
at a transformer, including an encoder including a plurality of encoder layers, and a decoder including a plurality of decoder layers, using a bi-linear multi-head attention layer, forming part of each layer of the encoder and decoder, to compute second-order interactions between vectors associated with the extracted image features;
using a positional encoder to provide contextual order to decoder bi-linear multi-head attention layer output; and
using a text-generation module to generate a text-based medical report of the image based on an output from the transformer.

13. The method of claim 12, further comprising:
using a bi-linear dot-product attention layer forming part of the bi-linear multi-head attention layer to produce one or more query vectors, key vectors and value vectors based on the extracted image features.

14. The method of claim 13, further comprising using the bi-linear multi-head attention layer to compute the second-order interaction between the produced one or more query vectors, key vectors and value vectors.

15. The method of claim 12, further comprising basing the positional encoder on periodic functions to describe relative location of medical terms in the medical report.

16. The method of claim 12, further comprising using an optimization module to perform recursive chain rule optimization of sentences in the text-based medical description.

17. The method of claim 12, further comprising using a tensor having a same shape as an input sequence as part of the positional encoder.

18. The method of claim 12, further comprising using one or more add and learnable normalisation layers to produce combinations of possibilities of resulting features of each of the bi-linear multi-head attention layers included in the encoder.

19. The method of claim 12, wherein the encoder receives two or more inputs containing feature representation from a plurality of image modalities.

20. The method of claim 12, further comprising using a search module configured to perform beam searching to further boost standardisation and quality of the generated medical reports.

21. (canceled)

22. (canceled)
